(19) United States
(12) Patent Application Publication
(10) Pub. No.: US 2007/0021805 A1
(43) Pub. Date: Jan. 25, 2007

(54) Biocompatible sensor electrode assembly and method for the production thereof

(76) Inventor: Bela Kelety, Frankfurt am Main (DE)

Correspondence Address: D. Peter Hochberg, 1940 East 6th St - 6th Floor, Cleveland, OH, 44114, US

(21) Appl. No.: 10/556,422

(22) PCT Filed: May 10, 2004
(86) PCT No.: PCT/EP04/04993

§ 371 Date: Nov. 9, 2005

(30) Foreign Application Priority Data
May 9, 2003 (DE) 103 20 898.4

(51) Int. Cl. A61N 1/00

(52) U.S. Cl. 607/62

(57) ABSTRACT

The invention relates to a biocompatible sensor electrode arrangement and to a process for its manufacture, at least one carrier substrate area (22), at least one intermediate substrate area (26) on the surface area (22a) of the carrier substrate area (22) and a biomaterial area (24) on the top side surface area (26a) of the intermediate substrate area (26) being provided. The biomaterial area (24) consists of at least one biologically compatible material component. The carrier substrate area (22) with the intermediate substrate area (26) is formed in the form or the manner of a wafer element or a printed circuit, as photolithographically processed structure, as structure bonded on or laminated on and/or as structure processed by printing, in particular on the carrier substrate (22) in each case.

### CROSS-REFERENCE TO RELATED APPLICATIONS

This application is a National Stage application of International Application No. PCT/EP2004/004993, filed on May 10, 2004, which claims priority of German application number 103 20 898.4, filed on May 9, 2003.

### BACKGROUND OF THE INVENTION

1. Field of the Invention

The invention relates to a biocompatible or biologically compatible sensor electrode arrangement and a process for its manufacture.

2. Description of the Prior Art

In many areas of chemical and biochemical analysis technology, biocompatible or biologically compatible material arrangements, in particular biocompatible or biologically compatible sensor arrangements or sensor electrode arrangements are used. As a result of these material arrangements, sensor arrangements or sensor electrode arrangements, certain measuring processes are used in practical application with respect to a chemical, biological or biochemically relevant analyte.

In the case of analytical processes with high rates of throughput, i.e. in the case of so-called high throughput screening processes, different characteristics are desirable for biocompatible material arrangements, sensor arrangements or sensor electrode arrangements, in particular with regard to their electrical sensitivity, their mechanically stability and/or their high and cost-effective availability. In the case of conventional material arrangements, sensor arrangements or sensor electrode arrangements, carrier substrates are used which, although having a mechanically relatively stable construction, they may entail an otherwise comparatively difficult handleability and also not be necessarily producible in a cost-effect manner.

### SUMMARY OF THE INVENTION

The invention is based on the task of indicating a biocompatible or biologically compatible sensor electrode arrangement and a process for its manufacture in the case of which cost-effective and both reliably handleable biocompatible or biologically compatible material arrangements are used in a particularly simple manner.

This task is achieved in the case of a biologically compatible or biocompatible sensor electrode arrangement according to the present invention. Moreover, the task is achieved according to the invention in the case of a process for the manufacture of a biologically compatible or biocompatible sensor electrode arrangement. The biologically compatible or biocompatible sensor electrode arrangement according to the invention exhibits at least one carrier substrate area which is formed with a top side with a surface area or a top side surface area. Moreover, an intermediate substrate area or a connecting substrate area is provided which is formed on the surface area of the carrier substrate area or a part thereof, in particular in a structured manner, and which is formed with a top side facing away from the carrier substrate area with a surface area or with a top side surface area. Finally, a biomaterial area is provided which is formed on the top side surface area of the intermediate substrate area or the connecting area or a part thereof, in particular in a structured manner, with at least one biocompatible or biologically compatible material component. The carrier substrate area with the intermediate substrate area or the connecting substrate area thereon or the intermediate substrate area or the connecting substrate area as such and/or a part thereof in each case are formed, for example, in the form or in the manner of a wafer element or a printed circuit. Alternatively or additionally, the intermediate substrate area or the connecting substrate area are formed as a or with a photolithographically processed structure or as a or with a photographically processed element. As a further alternative or additionally, the intermediate substrate area or the connecting substrate area are provided as a or with a structure processed by being bonded on or laminated on or as an or with an element processed by being bonded on or laminated on. As a further alternative or additionally, the intermediate substrate area or the connecting substrate area are formed as a or with a structure processed by printing or as an or with an element processed by printing. Moreover, alternatively or additionally, the intermediate substrate area or the connecting substrate area are formed as a or with a structure processed micromechanically and/or by laser ablation or as an or with an element processed micromechanically and/or by laser ablation.

This takes place in particularly directly on the carrier substrate area in each case.

It is consequently a core idea of the present invention to form the biocompatible sensor electrode arrangement according to the invention based on a carrier substrate area, between the carrier substrate area and the biomaterial area, an intermediate substrate area or connecting substrate area being provided by way of at least one biocompatible material component.

A further core idea of the present invention consists of the fact that the carrier substrate area with the intermediate substrate area or the connecting substrate area or that the intermediate substrate area or connecting substrate area as such or parts thereof are provided as a wafer element, as a printed circuit, as a photolithographically processed structure, as a structure bonded or laminated on, as structure processed micromechanically and/or by laser ablation and/or as a structure processed by printing, in particular on the substrate carrier area in each case.

As a result of this structure provided according to the invention, advantages arise compared with the state of the art to the effect that low material and manufacture costs, for example, arise in the case of the biocompatible sensor electrode arrangement according to the invention because common mass manufacture techniques connected with the suggested modes of formation and structures can be used during the manufacture.

Moreover, a corresponding mechanical strength of the biocompatible sensor electrode arrangement according to the invention is obtained automatically with a high reliability in use and a high application flexibility, the aspect of miniaturisation of the structures of the biocompatible sensor electrode arrangements of the invention being thereby taken into consideration, on the basis of the corresponding process technology, sufficiently and in a reliable manner.

Although it is possible to produce the intermediate substrate area or the connecting substrate area by epitaxial growing, by vapour deposition and/or sputtering in a particularly well controlled, well defined manner and/or with planar surfaces, it is possible under certain circumstances for straight structures and processes for their manufacture and corresponding techniques to offer themselves for reasons of costs and/or simplification, in the case of which such a control of the area properties and surface properties of the intermediate substrate area or the connecting substrate area is not possible, such as e.g. in the case of application and/or structurisation by photolithography, bonding, lamination, printing, ablation, laser ablation or such like if a good definition, controllability and/or planarity of the intermediate substrate area or the connecting substrate area or their surface are not important.

In the case of a preferred embodiment of the biocompatible sensor electrode arrangement according to the invention it is provided that the carrier substrate area exhibits a chemically inert, biologically inert and/or essentially electrically insulated material or is formed of such a material. As a result of this measure, minimal interactions with the chemical or biological surroundings arise and the use as a carrier for an electrode offers itself in particular.

With a view to the multiple application possibilities, it may be additionally or alternatively provided for the carrier substrate area to exhibit a mechanically flexible material or to be formed of such a material, in particular in the manner or in the form of a film. This then allows the manufacture of, e.g., single use sensors for single application, e.g. for diagnostic or clinical purposes or for use in the field, in situ or at the point of care.

With the regard to the structurisation of the biocompatible material arrangement as a sensor electrode arrangement, it is provided additionally or alternatively in the case of another embodiment of the sensor electrode arrangement according to the invention that a layer of an electrically conductive metal oxide, e.g. ITO or indium tin oxide is formed for the intermediate substrate area or for the connecting substrate area.

On the other hand, it may be possible, with respect to the structuralisation of the biocompatible material arrangement as a sensor electrode arrangement in the case of another embodiment of the sensor electrode arrangement according to the invention to be additionally or alternatively provided that a metallic layer structure is formed on the top side surface area of the carrier substrate area for the intermediate substrate area or for the connecting substrate area.

In this connection, it may be advantageous if the layer structure for the intermediate substrate area or for the connecting substrate area is formed with or from at least one primary metal area arranged at bottom most, a subsequent auxiliary layer and an actual electrode layer arranged top most.

The auxiliary layer can serve, in particular, as an alloy and/or diffusion barrier between the primary metal area and the actual electrode layer such that an alloy formation as a result of interdiffusion of the materials of the primary metal area and the actual electrode layer into or with each other is avoided. This maintains, for example, the specificity of the actual electrode layer, in particular in the case where a surface functionalisation or surface improvement of the actual electrode layer is involved.

The primary metal area can be formed with or of copper, for example.

The structurisation of the primary metal area can be provided in different forms, e.g. in the form of a photolithographically structured or applied primary metal area. In addition or alternatively, primary metal areas applied by bonding, lamination, ablation and/or processed by printing are conceivable.

According to another embodiment of the sensor electrode arrangement according to the invention, the auxiliary layer is formed with or from nickel.

In the case of a further alternative embodiment of the sensor electrode arrangement according to the invention it is provided that the actual electrode layer arranged top most is formed with or from a noble metal. Gold is preferably used in this respect.

The auxiliary layer and/or the actual electrode layer arranged top most can be formed by electrodeposition.

Alternatively or additionally, it is possible to consider a micromechanical formation and/or a laser ablation.

With respect to the further biological, chemical or biochemical compatibility of the sensor electrode arrangement as a whole, it can be provided that the carrier substrate area is formed entirely or partly from a chemically inert, biologically inert material and/or material with an at most lost adsorptivity vis-à-vis proteins, biological and/or chemical active substances.

With regard to the material selection, entirely different materials can be used in the carrier substrate area:

It is conceivable to use glass, quartz and/or mica as materials for or in the carrier substrate area because, then, a particularly well-defined, well-controlled and/or planar carrier substrate area and/or surface area is then present.

However, it is advantageous if the carrier substrate area is formed entirely or partly of PMMA, PEEK, PTFE, POM, FR4 polyimide such as e.g. PI or Kapton, PEN, PET and/or a material which is transparent—in particular in the UV range—if good definition, control and/or planarity of the carrier substrate area and/or the surface area is not important. As a result of this material selection, the processing effort and the costs can be reduced.

The meaning in this respect is as follows:

| | |
|---|---|
| PMMA | polymethylmethacrylate |
| PEEK | polyetheretherketone |
| PEN | polyethylene naphthalat |
| FR4 | glass fibre-reinforced epoxy resin |
| PI | polyimide |
| PET | polyethylene terephthalate |
| POM | polyoxymethylene and |
| PTFE | polytetrafluoroethylene or Teflon |

PEEK, POM and PTFE are not transparent as a rule. PTFE can usually not be bonded. However, if bonding techniques other than UV bonding are used, these materials can also be used in a meaningful manner.

With respect to as high a surface yield as possible and/or possible automation, it is, moreover, advantageous if a multiplicity, in particular of identical intermediate substrate areas or connecting substrate areas and/or corresponding biomaterial areas is formed. This plurality can be formed in a connected or in a separated form. In particular, an electrical insulation from each other offers itself in order to obtain sensor elements of the sensor electrode arrangement which are separated and insulated from each other, for example. The majority of intermediate substrate areas or connecting substrate areas and/or biomaterial areas is formed also in a form laterally arranged side by side on the carrier substrate area, for example.

An arrangement of the plurality of intermediate substrate areas or connecting substrate areas and/or corresponding biomaterial areas in a sequence or matrix form is particularly advantageous.

In a particularly advantageous manner, the use of the sensor electrode arrangement according to the present invention offers itself as sensor electrode arrangement for amperometric and/or potentiometric pharmacological effective site and/or active principle testing.

The intermediate substrate area and/or the biomaterial area or their combination can be provided in an advantageous manner as membrane biosensor electrode area or as secondary carrier of the sensor electrode arrangement.

The intermediate substrate area or the connecting substrate area and the biomaterial area can be formed respectively as a membrane biosensor electrode area or as secondary carrier with an electrically conductive and solid body-type electrode area.

With respect to the use in the area of amperometric and/or potentiometric pharmacological active site and/or active principle testing, in particular, further aspects may be essential as will be explained below.

In the case of the biocompatible or biologically compatible sensor arrangement or sensor electrode arrangement according to the invention for amperometric and/or potentiometric, pharmacological active site and/or active principle testing, a membrane biosensor electrode area is provided which, according to the meaning of the invention, is also referred to synonymously as secondary carrier. This membrane biosensor electrode area or secondary carrier exhibits an electrically conductive and solid body-type electrode area.

This is formed by the intermediate substrate area or the connecting substrate area, for example, and in particular by the actual electrode layer.

Moreover, a plurality of primary carriers is provided which are arranged in the immediate spatial vicinity of the membrane biosensor electrode area or the secondary carrier and which exhibit units which are activable to an electrical action and biological, in particular membrane proteins. In addition, an aqueous measuring medium is provided in which the primary carriers and at least part of the membrane biosensor electrode area or the secondary carrier are arranged.

According to the invention, the electrode area is formed in a manner that is electrically insulated vis-à-vis the measuring medium, the primary carriers and vis-à-vis the biological units.

According to the invention, a eukaryotic cell, a prokaryotic cell, a bacterium, a virus or components, in particular membrane fragments or associations thereof in the native form or in the modified form, in particular in the purified, microbiological form and/or form modified by molecular biology are provided as primary carrier in each case. Alternatively or additionally, a vesicle, a liposome or a micellar structure are provided as primary carrier.

An essential component of the sensor arrangement or sensor electrode arrangement according to the invention also consists of a membrane biosensor electrode area or a secondary carrier which, in the following, can also be called sensor electrode device. This sensor electrode device for amperometric and/or potentiometric, pharmacological active site and/or active principle testing itself thus exhibits at least one electrically conductive electrode area. The sensor electrode device is formed such as to be arranged in an aqueous measuring medium during operation. Moreover, the sensor electrode device is formed such that a plurality or a multiplicity of primary carriers with biological units activable into electrical action, in particular membrane proteins or such like are arranged in immediate spatial vicinity, in particular of the electrode area. According to the invention, at least the electrode area is in this respect formed like a solid body. Moreover, according to the invention, the electrode area is formed such that it is electrically insulated vis-à-vis the measuring medium to be provided and vis-à-vis the primary carriers.

It is thus a further idea of the present invention to form at least the electrode area of the sensor arrangement according to the invention and, in particular, the sensor electrode device as a solid body or in a solid body supported manner. As a result, the sensor electrode device and, in particular, the provided electrode area thereof are provided with a particularly high mechanical stability as a result of which a particularly robust operation not susceptible to interference is possible within the framework of the active site and/or active principle testing.

Only by solid body support is an activation e.g. of membrane proteins made possible by a concentration jump. This can take place, in particular, within the framework of a rapid and/or continuous solution exchange as a result of which—particularly in the case of amperometric measurements—a high signal level and consequently a high sensitivity can be achieved. As a result of the robustness due to the solid body support, easier handling and convenient incorporation are possible.

A further aspect of the present invention consists of forming the electrode area in such a way that it is formed in an electrically insulated manner vis-à-vis the measuring medium and vis-à-vis the primary carriers during operation. As a result of this measure it is possible to use the sensor electrode device as a capacitively coupled electrode, for example. This has considerable advantages particularly with regard to the signal-to-noise ratio, i.e. with regard to the accuracy of detection. Moreover, in the case of capacitive coupling, the electrode area of the sensor electrode device does not participate in any chemical conversion such as would be the case with a typical electrochemical half-cell.

The selection of the primary carriers carrying the biological units needs to be regarded as a further core aspect of the sensor arrangement according to the invention. The primary carriers may consist of eukaryotic cells, prokaryotic cells, bacteria, viruses or components, in particular membrane fragments or associations thereof respectively, namely in the native form or in a modified form, in particular in a purified form or a form modified by molecular biology and/or microbiologically. Alternatively or additionally, vesicles, liposomes or micellar structures are conceivable as primary carriers.

In the case of a particularly advantageous embodiment of the sensor arrangement according to the invention, it is anticipated that the electrode area exhibits at least one electrically conductive electrode, that an electrically insulating insulation area is provided and that the electrode concerned is electrically insulated from the measuring medium, from the primary carriers and from the biological units by the insulation area.

In this case, the electrode is formed by the intermediate substrate area or by the connecting substrate area, for example, and in particular by the actual electrode layer. The biomaterial area forms the insulation area in this case.

The electrode area also advantageously possesses at least one electrode. This can be formed, on the one hand, as such as a mechanically stable material area.

On the other hand, the electrode area can also exhibit a carrier which is formed in particular in the form of a solid body. This function is assumed by the carrier substrate area, for example. It is then possible for the electrode to be formed, respectively, as a material area or material layer on a surface area or the surface of this carrier, in particular in a continuous manner. In this respect, it is anticipated in particular for the electrode to achieve mechanical stability as a result of the solid body support provided by the carrier. This procedure has the advantage that, if necessary, high-value materials can be applied onto the carrier as a thin layer, for example, such that the possibility of a single use sensor electrode device presents itself in an economic operating respect, which device can be manufactured at affordable prices and utilised on the market. If necessary, the carrier, in particular the electrode area, can be recycled in which case, in particular, a replace insulation area, e.g. a new thiol layer, may become necessary.

Preferably, the electrode exhibits at least one metallic material or is formed of such a material. In this respect, a chemically inert noble metal, in particular, preferably gold, is advantageously used. Platinum or silver, in particular, are also conceivable.

Moreover, the use of electrically conductive metal oxides for or in the intermediate substrate area or the connecting substrate area, e.g. of ITO or indium tin oxide, is conceivable. From or with this class of material, counter-electrodes, which may need to be provided, may be manufactured.

The carrier for accepting the electrode consequently advantageously exhibits an electrically insulating material or is formed of such a material. Moreover, or alternatively, it is advantageous for the material of the carrier to be essentially chemically inert. Advantageously, glass or the like offers itself as a material. In this respect, the shape may that of a panel or such like. The chemical inertness prevents both a modification of the carrier and a contamination of the measuring medium during the measuring process. As a result of the selection of an electrically insulating carrier, it is guaranteed that all measuring signals originate essentially from the area of the electrode.

A possible arrangement of the sensor electrode device is obtained if the electrode is essentially formed as material layer deposited on the surface of the carrier. It can also be a vapour deposited or sputtered material layer. The material layer for the formation of the electrode has a layer thickness of approximately 10 nm to 200 nm, for example.

Between the material layer for the electrode and the surface of the carrier, an adhesive layer may, if necessary, be of advantage. On applying a gold electrode onto glass, in particular, an adhesive layer of chromium or such like present in between is of advantage. Advantageously, the adhesive layer has a relatively low layer thickness, preferably of approximately 5 nm.

To form the capacitive electrode and the insulation of the electrode area from the measuring medium and/or from the primary carriers, which is necessary for this purpose, at least one insulating area or biomaterial area is thus preferably formed as a result of which the electrode area, in particular the electrode, is essentially electrically insulatable in operation, in particular in areas thereof which are provided for mechanical contact with the measuring medium and/or the primary carriers during operation.

In a further preferred embodiment of the sensor arrangement or sensor electrode arrangement according to the invention, the insulation area or biomaterial area is formed in the form of layers. In this respect, the insulation area or biomaterial area consists at least partly of a sequence of monolayers, the monolayers being formed as spontaneously self-organising layers.

In this respect, it is advantageous for a layer of an organic thio compound to be provided as a sub-layer of the insulation area or biomaterial area or as a bottom most area, or an area facing towards the electrode, of the insulation area or the biomaterial area, with a view to the electrical properties and the electrical insulation, preferably of a long-chain alkane thiol, in particular of octadecane thiol.

Moreover a layer of an amphiphilic organic compound, in particular of a lipid, is provided as top layer of the insulation area or biomaterial area, as an uppermost area facing away from the electrode or surface area of the insulation area.

It can thus be advantageous to form the insulation area or biomaterial area at least partly in layer form, in particular in multilayer form. In this way, the insulation effect is strengthened and the manufacture simplified. In order to obtain as high a rate of attached and/or arranged primary carriers as possible in the area of the sensor electrode device, it is anticipated according to a preferred embodiment of the sensor electrode device according to the invention that at least the surface area of the insulation area is formed in such a matched manner that an attachment and/or arrangement of primary carriers on the surface area of the insulation area is promoted, in particular in a manner compatible with the surface of the primary carriers. This means that, depending on the surface properties of the primary carrier, the surface area of the insulation area of the sensor electrode device be formed in a correspondingly adjusted manner such that the primary carriers attach themselves in a favoured manner to the surface area of the insulation area and remain there.

With respect to a particularly marked capacitive coupling of the sensor electrode device during measuring operation, it is therefore anticipated that the insulation area is formed at least partly as a single layer, monolayer and/or as a sequence thereof. In this case, the specific area-related electric capacitance of the electrode boundary layer is particularly high. The arrangement and formation of the sensor arrangement according to the invention is particularly simple if the layer or layers of the insulation area are formed as spontaneously self-organising layers or as self-assembling layers. In this respect, the tendency and the endeavours of certain essentially liquid starting materials or those dissolved in the liquid state to form, on a surface, under the influence of the interaction with the structure of the surface, spontaneously and in a self-organising manner, an ordered and/or layer-type structure which, under certain circumstances and in the case of certain classes of substances leads to the formation of particularly thin and, if necessary, single-layer layers or monolayers, in particular of molecules, are exploited in an advantageous manner.

In the case of the use of, according to the invention, organic thio compounds, in particular of alkane thiols, use is made of the fact that, on certain noble metal surfaces, e.g. gold, silver and platinum, it is possible to form, from an organic solution which contains the corresponding thio compound in solution, a covalently bonded monolayer on the electrode surface as a result of a specific chemical interaction of the thio group with the surface atoms of the noble metal electrode, which monolayer is capable of forming a hexagonal dense package in the case of a corresponding geometry of the thio compound, as a result of which a particularly low residual conductivity of the noble metal surface is achievable with respect to the measuring medium to be provided.

Correspondingly, it is possible when using metal oxides in the area of exposed surface areas of the intermediate substrate area or the connecting substrate area, in particular of indium tin oxide, to make use of a correspondingly specific siloxane chemistry for the formation of a covalently bonded sub-layer of the insulation area or the biomaterial area, in which, as top layer or as uppermost layer and area facing away from the electrode or surface area of the insulation area or biomaterial area, a layer of an amphiphilic organic compound, in particular a lipid and/or the like is provided.

As a result of the procedure in which, as top layer or as uppermost area facing away from the electrode or surface area of the insulation area or biomaterial area, a layer of an amphiphilic organic compound, in particular a lipid and/or the like is provided, a particularly well-defined arrangement and structurisation of the surface of the insulation area or biomaterial area is forcefully obtained.

The amphiphilic organic compounds possess at least one area of polar formation such that a certain partial solubility arises in the measuring medium which, in particular, is of an aqueous nature. On the other hand, amphiphilic organic compounds possess a non-polar or hydrophobic area whose arrangement in an aqueous measuring medium is less preferred from the energy point of view. As a result of this phenomenon, a layer structure is preferably formed in the case of which the polar or water-soluble areas of the amphiphilic compounds are allocated to the aqueous measuring medium whereas the non-polar or hydrophobic areas of the amphiphilic organic compounds are arranged facing away from the aqueous measuring medium. Consequently, a monolayer can be formed which forms, in particular, the surface area of the electrode area. This is preferably done in combination with an alkane thiol monolayer as sub-layer such that, at least partly, a double layer of two monolayers is formed as insulation area or biomaterial area.

The sequence of two monolayers thus formed has certain structural similarities to certain membrane structures which are known from biological systems such that a certain membrane structure can be allocated to the sequence of two monolayers thus formed—namely the alkane thiol monolayer facing towards the electrode and the lipid monolayer arranged on top. As a result of the basic solid body carrier, this membrane structure is also referred to as solid body supported membrane SSM (SSM: solid supported membrane). This SSM membrane structure has particularly advantageous properties with respect to the arrangement and characteristic property of the sensor electrode device according to the invention, as a capacitively coupled electrode.

The area which is defined by the electrode-insulating and/or covering layer of the insulation area or biomaterial area, in particular, exhibits the membrane structure just described in an advantageous manner. In this respect, it is also advantageous that this membrane structure or SSM has at least in part a specific electric conductivity of approximately Gₘ≈1-100 nS/cm². Moreover, a specific electric capacitance of approximately Cₘ≈10-1000 nF/cm² is advantageously present. Finally a surface for the membrane structure of approximately A≈0.1-50 mm² is provided alternatively or as a supplement.

The high specific capacitance Cm is of particular advantage with respect to an amperometric active principle test to be carried out, in the case of which initiated electrical actions of the essentially biological units are measured as electric currents, namely as displacement currents or capacitive currents.

With a view to the signal-to-noise ratio, a corresponding sealant resistance in the area of a few nanosiemens is of particular advantage.

According to another embodiment of the sensor arrangement according to the invention, this can also be achieved by applying a Teflon layer, e.g. directly onto the metal electrode. Such a procedure is entirely sufficient for potentiometric active principle testing, for example, since, in this case, it is not a high electrical capacitance which is important but a high sealing resistance because of the voltage measurements.

Particularly simple geometric circumstances arise, in particular with a view to the reproducibility of the measured results, if the carrier, the electrode and/or the insulation area and/or its surface or boundary surface areas are formed at least partly in an essentially planar manner, in particular also at the microscopic level or scale. The planarity guarantees that certain field strength effects at the edges or tips which may lead to the breakthrough of the sealing resistance, do not arise. Moreover, with a view to the exchange, in operation, of the measuring medium to be provided, the advantage of a homogeneous boundary surface distribution arises. Any possible protuberances or cavities would lead to concentration inhomogeneities at the boundary surface between the insulation area and the measuring medium, which inhomogeneities could possibly have a negative influence on the results of detection or measurement achieved. The planarity, in particular of the metallic boundary surfaces, can be guaranteed by corresponding manufacturing processes, e.g. by epitactic growth, annealing or such like.

For external contacting of the sensor arrangement, e.g. with an external measuring circuit or the like, a contact area is provided, a corresponding insulation to avoid other short circuits, in particular with respect to the measuring medium, being formed.

In particular with a view to a high rate of throughput in the case of active site and/or active principle tests to be carried out correspondingly, it is particularly advantageous if the sensor arrangement according to the invention is formed in such a way that, at least in operation, it exhibits essentially constant mechanical, electrical and/or structural properties vis-à-vis liquid streams with a high flow rate, preferably in the region of approximately v≈0.1-2 m/s, in particular in the region of the membrane structure and/or especially with a view to the attachment and/or arrangement of primary carriers. This required and advantageous consistency of the mechanical, electrical and/or structural properties of the sensor arrangement according to the invention and, in particular, the membrane structure provided therein is obtained inherently as a result of the above-mentioned measures for the formation of the electrode and the insulation layer covering the electrode, in particular in the form of self-assembling monolayers of an alkane thiol on gold with a corresponding monolayer of lipid in an aqueous medium.

Advantageously, the sensor arrangement according to the invention is used with the sensor electrode device described, in a process for amperometric and/or potentiometric, pharmacological active site and/or active principle testing and in a device for carrying out such a process.

In the case of the sensor arrangement according to the invention, a eukaryotic cell, a prokaryotic cell, organelles thereof, a bacterial unit, a viral unit and/or such like and/or components, fragments, in particular membrane fragments of such like and/or associations thereof in an essentially native and/or modified, in particular purified form or form modified microbiologically and/or by molecular biology are provided as primary carriers respectively.

It is thus conceivable in principle that insulated and whole cells are used as primary carriers of corresponding biological units which can be activated to an electrical action, irrespective of whether these are of plant or animal origin. Thus, an examination of entire heart cells, for example, is possible and conceivable. On the other hand, the examination of plant cells, for example algae cells or other unicellular organisms, can also be considered. In addition, certain bacteria or viruses can be examined as a whole. Moreover, it is conceivable to use components or fragments of cells, bacteria or viruses as primary carriers as a result of specific microbiological or biochemical measures. Also, it is conceivable to use associations of cells, bacteria or such like as primary carriers and to connect these to the corresponding sensor electrode device for the formation of a sensor arrangement according to the invention.

Moreover, the possibility exists according to the invention of using the suggested primary carriers in their native form or in a modified form. In this respect, eukaryotic cells, prokaryotic cells or bacteria, for example can be used which have been modified by corresponding purification, microbiological and/or molecular biology processes in order to preferably form specific proteins with certain desired properties, for example.

Apart from the primary carriers already available in their natural form in the form of cells, bacteria and the like, it is also conceivable to produce artificial primary carriers in the form of vesicles, liposomes, micellar structures and/or the like, for example. If necessary, these are then provided and/or enriched with corresponding biological units which can be activated to electrical action. Corresponding processes for the reconstitution of membrane proteins or such like in vesicles or liposomes are known and can be exploited here in an advantageous manner in order to create particularly advantageous embodiments of the sensor arrangement according to the invention.

Suitable as essentially biological units are all units which can be triggered into an at least partly electrically produced action. Such biological units are conceivable in particular which are activable to perform an at least partial electrogenic and/or electrophoretic charge carrier transport and/or an at least partial electrogenic or electrophoretic charge carrier movement and which represent biological, chemical and biochemical units. These are in particular transport units which move charge carriers upon their activation. Components, fragments and/or associations of such units, in particular transport units, are also conceivable.

Membrane proteins, in particular ion pumps, ion channels, transporters, receptors and/or such like offer themselves in particular as biological units. With respect to many of these biological units, findings and/or assumptions exist to the effect that certain processes are associated with at least one electrogenic partial step. These electrogenic partial steps can be associated with an actual substance transport such as in the case of a channel, an ion pump or certain transporters, for example. However, biological units, in particular membrane proteins, are also known whose electrical activity is not connected with a net material transport but rather with a, if necessary reversible, charge displacement within the framework of a conformation change or bonding or such like. Such electrical activities, too, are measurable, in principle, according to the invention as short-term displacement currents and/or potential changes.

The biological units, in particular the membrane proteins, can be provided in essentially their native form and/or in a modified, in particular purified form or a form modified microbiologically and/or by molecular biology, respectively. On the one hand, certain native properties, can be tested and pharmacologically investigated in the organism of existing proteins, for example. On the other hand, modifications initiated by molecular biology or gene technology also offer themselves for analysing certain aspects, e.g. the transportation or the pharmacological mode of action of an active principle.

It is particularly advantageous that primary carriers of an essentially uniform type of primary carrier are provided in each case. This is of importance with regard to as unambiguous as possible as evidence and analysis of an active substance test and relates to the geometric, physical, chemical, biological and molecular biological properties of the primary carrier.

The same also applies to the biological units provided for the primary carrier, in particular to the membrane proteins or such like. In this case, biological units of an essentially uniform type are provided in each case, in particular with respect to their geometrical, physical, chemical, biological and molecular biological properties. In addition, the biological units should advantageously be approximately uniform with respect to their orientation and/or with respect to their activatibility in relation to the primary carrier concerned.

To achieve as high a signal quality as possible, it is advantageous for the surfaces of the primary carrier and/or the secondary carrier to be formed in such a way that an attachment and/or arrangement of the primary carriers on the secondary carrier is promoted. In this way, a particularly high number of attached primary carriers and/or a particularly close contact of the primary carriers to the secondary carrier is obtained, on the one hand, as a result of which the electrical connection and consequently the signal-to-noise ratio are increased.

The attachment can be controlled e.g. via the so-called lipid-lipid interaction between the primary carrier, e.g. vesicle, and the secondary carrier, e.g. lipid thiol SSM. On the other hand, a covalent bond of the primary carrier to the surface of the secondary carrier is conceivable, e.g. in the form of a biotin-streptavidin scheme or according to the meaning of His-Tag coupling.

In this connection, it is particularly advantageous if the surfaces of the primary carriers and of the secondary carrier are formed with an opposite polarity to each other. This promotes the rate of attachment of the primary carriers to the secondary carrier and the strength of the contact between them.

It is particularly advantageous if vesicles or liposomes with essentially the same effect and/or of the same type, preferably of a lipid, are provided as primary carriers in and/or on the membrane of which units of essentially one type of membrane protein are embedded and/or attached in preferably essentially an oriented form.

The sensor arrangement according to the invention is advantageously used in a process for amperometric and/or potentiometric, in particular pharmacological active site and/or active principle testing and/or in a device for carrying out such a process.

According to a further aspect of the present invention, a process for manufacturing a biologically compatible or biocompatible sensor electrode arrangement, in particular a sensor electrode arrangement for amperometric and/or potentiometric, pharmacological active site and/or active principle testing is created.

In the manufacturing process according to the invention, at least one carrier substrate area with a top side with a surface area or with a top side surface area is formed. Moreover, at least one intermediate substrate area or a connecting substrate area is formed on the surface area or the top side surface area of the carrier substrate area or a part thereof, in particular in a structured manner and with a top side facing away from the carrier substrate area with a surface area or with a top side surface area. Moreover, a biomaterial area is formed on the top side surface area of the intermediate substrate area or the connecting substrate area or a part thereof, in particular in a structured manner, with at least one biologically compatible or biocompatible material component. According to the invention, the carrier substrate area with the intermediate substrate area or the connecting substrate area thereon or the intermediate substrate area or the connecting substrate area as such and/or a part thereof in each case are formed in the form or the manner of a wafer element or a printed circuit. Alternatively or additionally, it is anticipated that the carrier substrate area with the intermediate substrate area and the connecting substrate area thereon or the intermediate substrate area or the connecting substrate area as such and/or a part thereof in each case are formed as a or with a photolithographically processed structure or as a or with a photographically processed element, as a or with a structure processed by being bonded on or laminated on or as an or with an element processed by being bonded on or laminated on, as a or with a structure processed micromechanically and/or by laser ablation or as an or with an element processed micromechanically and/or by laser ablation and/or a structure processed by printing or as an or with an element processed by printing. This is provided in particular on the carrier substrate area in each case.

Basic aspects of the manufacturing process according to the invention consequently need to be seen in the fact that a carrier substrate area, an intermediate substrate area or a connecting substrate area thereon and a biomaterial area are provided on the surface area of the intermediate substrate area or the connecting substrate area. In this connection, it is a further aspect that the carrier substrate area with the intermediate substrate area or the connecting substrate area thereon or the intermediate substrate area or the connecting substrate area as such and/or a part thereof in each case are processed in a manner possible for wafers or printed circuits in order to achieve a particularly reliable and cost-effective manufacture, in particular in mass manufacture.

The further manufacturing modes with a view to the provision of photolithographically processed structures or elements, bonded-on and/or laminated-on processed structures or elements, structures or elements processed micromechanically and/or by laser ablation and/or structures or elements processed by printing need to be additionally or alternatively provided.

The ablation and/or laser ablation takes place, if necessary, with mask support.

The individual process steps of the manufacturing process according to the invention and/or their modifications are also carried out in line with the structural measures described above.

Thus, it is anticipated according to a preferred embodiment of the manufacturing process according to the invention that the carrier substrate area is formed with a chemically inert, biologically inert and/or essentially electrically insulated material or of such a material.

Moreover, it is anticipated alternatively or additionally that the carrier substrate area is formed with a mechanically flexible material or of such a material, in particular in the form or in the manner of a film.

With regard to the structurisation of the biocompatible material arrangement as sensor electrode arrangement, it is additionally or alternatively provided in the case of an another embodiment of the manufacturing process according to the invention of a sensor electrode arrangement that, for the intermediate substrate area or for the connecting substrate area, a layer of an electrically conductive metal oxide, for example ITO or indium tin oxide, is formed.

In the case of another embodiment of the manufacturing process according to the invention, it is anticipated that, for the intermediate substrate area or for the connecting substrate area, a metallic layer structure is formed on the top side surface or the top side surface area of the carrier substrate area.

In this connection, it may be anticipated in an advantageous manner that the layer structure for the intermediate substrate area or for the connecting substrate area is formed with at least one or of a primary metal area arranged bottom most, a subsequent auxiliary layer and an actual electrode layer arranged top most.

In this connection, the primary metal layer or the primary metal area is formed as an alloy barrier and/or diffusion barrier.

It is moreover preferred that the primary metal area is formed with or of copper.

Moreover, it is preferred that the primary metal area is formed photolithographically. Alternatively or additionally, the possibility offers itself to process by bonding on, laminating on, ablation and/or printing on.

The auxiliary layer is advantageously formed of nickel or containing nickel.

According to a further alternative embodiment of the manufacturing process according to the invention, the actual electrode material arranged top most or the actual electrode layer arranged top most is formed with or of noble metal, preferably with or of gold.

It is particularly preferred that the auxiliary layer and/or the actual electrode layer arranged top most is formed by electrodeposition.

Alternatively or additionally, micromechanical processing and/or laser ablation can be used.

Particularly advantageous properties of the sensor electrode arrangement to be manufactured are obtained if, according to a preferred embodiment of the manufacturing process, the carrier substrate area is formed entirely or partly of a chemically inert, biologically inert material and/or a material that is at most slightly absorptive vis-à-vis proteins, biological and/or chemical active principles.

In the case of a further advantageous embodiment of the manufacturing process according to the invention it is anticipated that the carrier substrate area is formed entirely or partly of PMMA, PTFE, POM, FR4, polyimide such as e.g. PI or Kapton, PEN, PET and/or of a material that is transparent—particularly in the UV range.

For further flexibilisation and enlargement of the area of use of the product to be manufactured according to the meaning of the sensor electrode arrangement to be manufactured, it is anticipated in the case of a further advantageous development of the manufacturing process according to the invention that a plurality—in particular of homogeneous—intermediate substrate areas or connecting substrate areas and/or biomaterial areas is formed. These may be formed in a connecting or in a separate form, in particular with a view to their electrical connection and/or electrical insulation with and/or from each other. This takes place, in particular, in a laterally separated manner.

In an advantageous manner, the plurality of intermediate substrate areas or connecting substrate areas and/or biomaterial areas is arranged in series or in matrix form.

According to a further advantageous embodiment of the manufacturing process according to the invention, the sensor electrode arrangement is formed as a sensor electrode arrangement for amperometric and/or potentiometric, pharmacological active site and/or active principle testing.

In this connection, the intermediate substrate area and/or the biomaterial area are provided in each case as membrane sensor electrode area or as secondary carrier of the sensor electrode arrangement.

In the case of another embodiment of the process according to the invention, the intermediate substrate area or the connecting substrate area and the biomaterial area are formed, in each case, as a membrane biosensor electrode area or as a secondary carrier with an electrically conductive and solid body-type electrode area.

In this connection, a plurality of primary carriers is provided in the immediate spatial vicinity of the secondary carriers or the secondary carrier. In this case, the primary carriers contain, biological units activable into electrical action, in particular membrane proteins.

According to the invention, a eukaryotic cell, a prokaryotic cell, a bacterium, a virus or components, in particular membrane fragments or associations thereof in the native form or in the modified form, in particular in the purified, microbiological form and/or form modified by molecular biology are provided as primary carrier in each case. Alternatively or additionally, a vesicle, a liposome or a cellular structure are provided as primary carrier.

Moreover, it may be anticipated that the intermediate substrate area or the connecting substrate area is provided as at least one electrically conductive electrode of the electrode area, that the biomaterial area is provided as an electrically insulated insulation area and that, in operation, the electrode concerned is electrically insulated by the biomaterial area or the insulation area from a measuring medium, from the primary carriers and from the biological units.

According to a further embodiment of the process according to the invention, it is anticipated that the biomaterial area or insulation area is formed in layers, that the insulation area is formed at least partly of a sequence of monolayers and/or that the monolayers are formed as spontaneously self-organising layers.

In the case of another embodiment of the process according to the invention, it is anticipated that, as a sub-layer of the biomaterial area or the insulation area, a layer of an organic thio compound is provided as a bottom most area of the insulation area facing towards the electrode, preferably of a long-chain alkane thiol, in particular of octadecane thiol, and that, as top layer of the biomaterial area or the insulation area, a layer of an amphiphilic organic compound, in particular of a lipid, is provided as uppermost area facing away from the electrode or surface area of the insulation area.

In the case of a further advantageous embodiment of the manufacturing process according to the invention, it is anticipated that the area of the biomaterial area or the insulation area insulating and covering the electrode is formed with a membrane structure with a surface of approximately A≈0.1-50 mm² and with a specific electric conductivity of approximately Gₘ≈1-100 nS/cm² and/or with a specific capacitance of approximately Cₘ≈10-1000 nF/cm².

According to a further preferred embodiment of the manufacturing process according to the invention, it is anticipated that a biological unit is provided which is formed to be activable to an electrogenic charge carrier movement, in particular to an electrogenic charge carrier transport.

Additionally or alternatively, it is anticipated that a membrane protein, in particular an ion pump, an ion channel, a transporter or a receptor or a component or an association thereof is provided as biological unit in each case.

Moreover, it is preferred alternatively or additionally that the biological unit is provided in native form or in a modified form, in particular in a purified, microbiologically modified form and/or a form modified by molecular biology.

In a further advantageous development of the manufacturing process according to the invention, it is anticipated that the surface of the primary carriers and the surface of the secondary carriers are formed with opposite polarity or oppositely charged to each other and/or that, between the surface of the primary carriers and the surface of the secondary carrier, a connection in the manner of a chemical bond is formed, in particular via a His-Tag coupling or a streptavidin-biotin coupling or the like.

These and other aspects of the present invention result, in other words, also from the following remarks:

The invention relates not only to corresponding structures but also to a process for the manufacture of electrically insulating, extremely thin layers as biocompatible areas or material areas, in particular on printed circuit boards or the like and to their use as sensor elements, in particular for single use.

In the field of bioanalysis, it is desirable in certain cases to have biocompatible surfaces available which are suitable for the absorptive attachment of biological membranes, membrane fragments or of artificial lipid double layers. The task, on which the invention described herein is based, consisted of manufacturing such surfaces in as cost-effective a manner as possible without suffering restrictions in functionality.

Methods are known which operate on the basis of optical measured values on biocompatible layers, so-called biacore measurements, for example, according to the principle of surface plasmon resonance or measurements of the load increase change using the quartz microbalance. In other cases, electrical properties of the attached, often protein-containing vesicles, cells or membrane fragments are to be detected.

Frequently, substrates or carrier substrate areas of mica, glass or quartz can be used which are coated e.g. with gold, by thin layer technique.

Occasionally, the selection of the substrate needs to be made on the basis of the specific properties of the substrate, e.g. glass, because of its transmittance in the area of visible light and because of its refractive index, quartz as a result of its ability to be induced to oscillation in the condenser field. Occasionally, glass is used because of its chemical inertness and the possibility of lithographic structuring of the gold layer down into the microstructure region.

Providing mica, glass and/or quartz with the intermediate substrate area or connecting substrate area by epitaxial growth, by vapour deposition and/or sputtering, for example, is meaningful and anticipated according to the invention in those cases where the controllability, high definition, high value and/or planarity of the intermediate substrate area or connecting substrate area and/or the corresponding surface areas are of importance.

It is also conceivable that areas can be produced on gold surfaces which are capable of providing specific bonding for target molecules.

The manufacture of structured biocompatible areas on gold surfaces which, in turn, have been applied onto glass substrates is complicated and costly under certain circumstances. Moreover, glass is fragile and may form, under certain circumstances, sharp edges capable of causing injuries.

These properties lead to the replacement according to the invention of glass substrates, mica or quartz as carrier for biocompatible areas according to the meaning of the invention by other materials and/or coating techniques other than epitaxial growing, vapour deposition and/or sputtering.

The use of a self-organising monolayer, a self-assembled monolayer or an SAM does not lead to the desired or necessary electrical properties and only partly to the ability of the surface to adsorb vesicles, cells or membrane fragments.

It is a core idea of this invention to produce, on printed circuit boards or such like producible cost-effectively in very large numbers, for example, biocompatible areas which provide a very low electric conductivity between the actual electrode or intermediate substrate and the surroundings and exhibit suitable adsorption properties with respect to cells, cell membrane fragments, liposomes or such like.

The structurisation of the printed circuit boards or such like takes place by selective or structured coating of a primary metal layer, e.g. a copper layer, for example, by subsequently selectively removing the primary metal layer, e.g. by wet-chemical etching, and by subsequent finishing, e.g. by gold plating.

These techniques permit the manufacture of very large numbers of items, the costs being lower by a multiple than those in the case of glass substrates. Moreover, biocompatible areas serving as biosensor can be manufactured in the immediate spatial vicinity to amplifier devices on wafers such that noise and interference can be considerably reduced. The substrates used for the manufacture are highly stable and, optionally, do not have sharp edges. They are therefore highly suitable for manufacturing disposables.

A copper-coated printed circuit board—e.g. with 17 μm copper—, for example, is coated with photoresist. A layout is transferred onto the photoresist by light exposure. The photoresist is developed and removed specifically in the areas not exposed to light, the copper layer being exposed in those areas. The copper layer is removed at the exposed sites. The remaining resist residues are also removed. Nickel, for example, is electrodeposited onto the free copper structures. Gold, for example, is electrodeposited onto the nickel layer thus obtained.

An alkane thiol monolayer is produced on the gold layer as self-assembled monolayer or SAM. By adding lipid-containing solution, a hybrid lipid layer is produced on the SAM in a manner analogous to a lipid double layer. This hybrid lipid layer permits the stable adsorption e.g. of membrane fragments of biological membranes, cell fragments, vesicles and liposomes.

By integrating the printed circuit boards or the like into an electric amplifier circuit and by integrating the biocompatible area into a flow cell and by introducing an Ag/AgCl reference electrode into the fluid-coupled system and by attaching membrane fragments with electrogenic membrane proteins, the modified printed circuit boards can be used as biosensors.

Further aspects of the present invention arise as follows:

Frequently, glass and/or a complicated technical process are used in order to obtain thin, very high quality intermediate substrate areas, connecting substrate areas and/or actual electrode layers according to the meaning of the invention, in particular gold layers, with it. The basic idea has been that a slightly rough surface is particularly advantageous.

This process may be uneconomical. As an alternative, it is thus possible to use intermediate substrate areas, connecting substrate areas and/or actual electrode layers, i.e. gold layers, for example, and corresponding surface areas of comparatively extremely poor quality with respect to visible granularity under the light microscope, layer thickness of several micrometers, beads, scratches etc, for example.

However, it has been found that essential characteristics of the membrane biosensor electrode area, i.e. the SSM, are retained such that the materials mica, glass, quartz and/or the application or structurisation by epitaxial growing, by vapour deposition and/or by sputtering are not necessarily required.

Consequently, fields of application for these comparatively low value but also cheap electrodes thus specifically arise in an advantageous manner.

Consequently, comprehensive extensions of the comparatively complex arrangements and the corresponding manufacturing processes thus arise according to the invention.

For this reason, too, the possibility offers itself to structurise gold-vapour deposition treated films, for example, consisting of polyimid or PEN, for example, by laser ablation, the laser beam being passed through a mask. The film may be drawn from a roll and structured in a continuous process.

### BRIEF DESCRIPTION OF THE DRAWINGS

In the following, the present invention is explained in further detail by way of a diagrammatic representation based on preferred practical examples.

FIG. 1 shows a diagrammatic and sectional side view of an embodiment of the biocompatible sensor electrode arrangement according to the invention.

FIGS. 2A, B show a diagrammatic top view and/or a sectional side view of another embodiment of the biocompatible sensor electrode arrangement according to the invention.

FIG. 3 shows a diagrammatic top view of a further embodiment of the biocompatible sensor electrode arrangement according to the invention with a plurality of individual electrodes.

FIG. 4 shows a diagrammatic and partial sectional side view of another embodiment of the biocompatible sensor electrode arrangement according to the invention with a vesicle as primary carrier and its use in a measuring device.

FIG. 5 shows a further embodiment of the biocompatible sensor electrode arrangement according to the invention with a membrane fragment as a primary carrier.

### DETAILED DESCRIPTION OF THE INVENTION

In the following the same references indicate the same, identical or identically acting structures or elements. A detailed description will therefore not be repeated each time they occur.

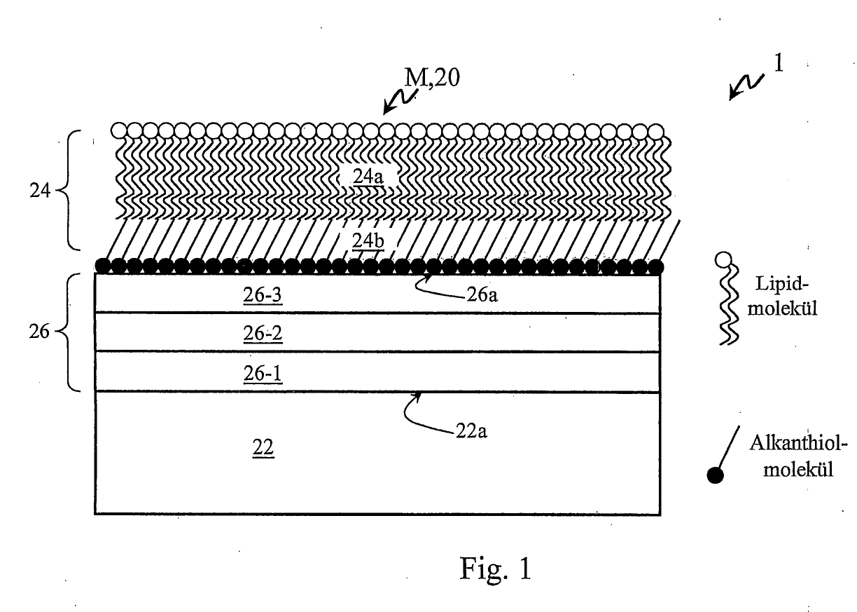

FIG. 1 shows a diagrammatic and sectional side view of a first embodiment of the biocompatible sensor electrode arrangement 1 according to the invention.

This first embodiment of the biocompatible sensor electrode arrangement 1 according to the invention exhibits a carrier substrate area 22 or a carrier substrate 22 with a top side surface area 22a on which the connecting intermediate substrate area 26 or the connecting substrate area 26 is provided in the form of a layered metal structure, namely with a primary metal area 26-1, of copper in this case, an auxiliary layer 26-2, e.g. of nickel in this case, which serves as diffusion barrier and alloy formation barrier, as well as an actual electrode layer 26-3, of gold in this case.

By a specific chemical interaction with the actual electrode layer 26-3, a biomaterial layer 24 or a biomaterial area 24 is immobilised on the top side surface area 26a of the connecting substrate area 26. This biomaterial area 24 serves as insulation area 24 for the sensor electrode arrangement 1 according to the invention and consists of a layered sequence of self-organising monolayers 24a and 24b, namely of a sub-layer arranged bottom most in the form of an alkane thiol monolayer 24b which is connected via the specific thiol gold interaction or SH—Au interaction, and a lipid monolayer 24a provided uppermost. By means of this arrangement, a membrane biosensor electrode device M or 20 with a solid body-supported membrane SSM is formed.

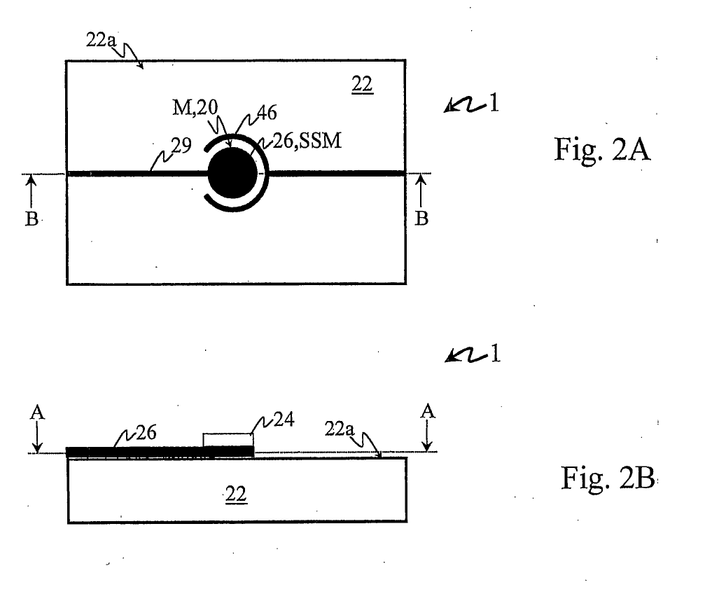

FIGS. 2A and 2B show a diagrammatic top view and/or a diagrammatic and sectional side view of another embodiment of the biocompatible sensor electrode arrangement 1 according to the invention. In this case, a processed counter-electrode device 46 is also shown in the top view of FIG. 2A, which device, however, was left out from the side view of FIG. 2B. This counter-electrode 46 can also consist of ITO or indium tin oxide and assume alternative embodiments.

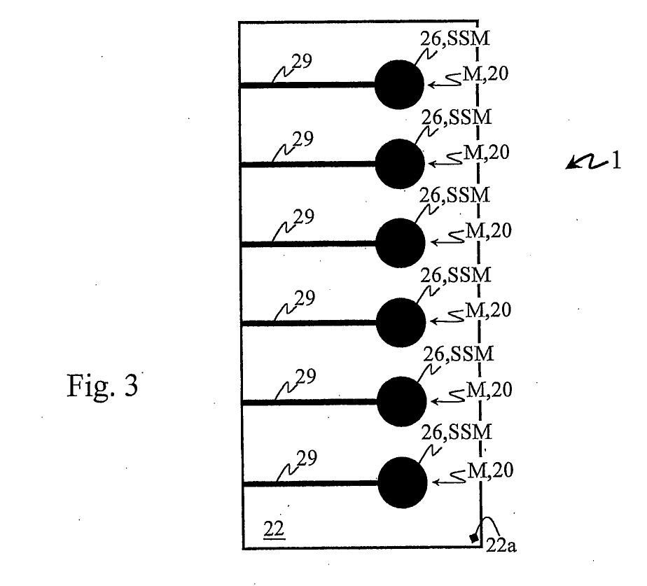

FIG. 3 shows, by way of a diagrammatic top view, an embodiment of the sensor electrode arrangement 1 according to the invention on which six individual electrodes 26 with corresponding supply leads 29 are formed on the upper surface 22a of the carrier substrate 22. The individual electrodes 26 with their corresponding terminal leads 29 are formed in an essentially identical manner, at least insofar as the manufacturing tolerances allow.

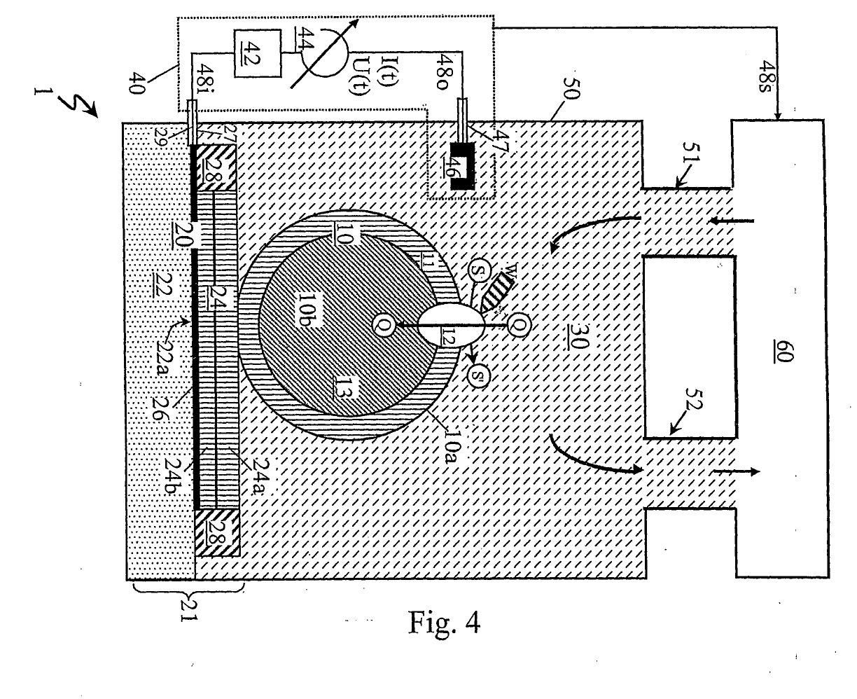

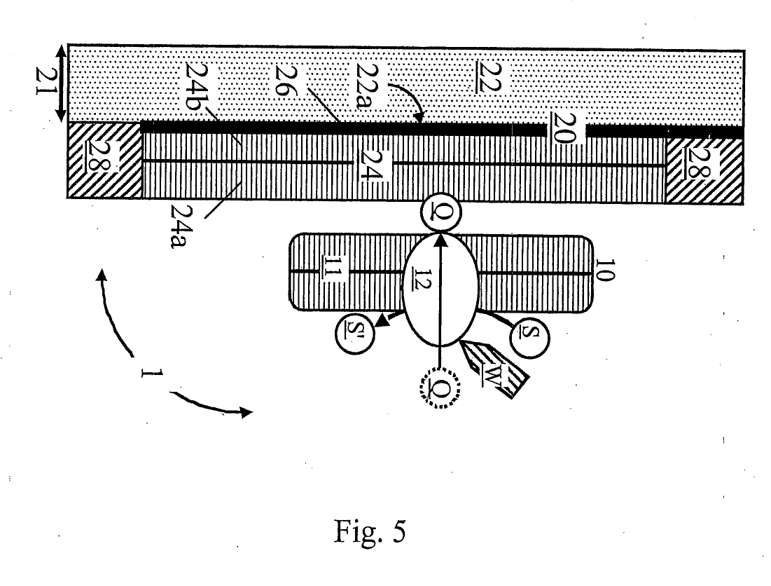

All characteristic properties relating to the mesoscopic or microscope structure of the surface of the membrane biosensor electrode area M, the secondary carrier 20 and, in particular, the respective allocated electrodes 26 can also be seen in the representation of the following FIGS. 4 and 5. All the characteristic properties illustrated therein are applicable in any random combination to the structures described above in FIG. 1 to 3.

FIG. 4 shows a diagrammatic and partly sectional side view of a further embodiment of the sensor arrangement 1 according to the invention and a corresponding device for amperometric and/or potentiometric pharmacological active principle testing.

A measuring chamber 50 in the form of an essentially closed vessel forms, together with an exchanger/mixing device 60 in the form of a perfuser system or a pump facility, for example, a closed liquid circuit. Communication of the liquid serving as measuring medium 30 is effected via corresponding feed and discharge devices 51 and/or 52. The measuring medium 30 can be an aqueous electrolyte solution in this case which exhibits certain ion moieties, a given temperature, a specific pH etc. Moreover, specific substrate substances S and/or specific active principles W are, if necessary, contained in the measuring medium 30 or they are added in later process steps through the exchange/mixing device 60.

In the measuring area 50, a sensor arrangement 1 according to the invention is provided. The sensor arrangement 1 consists of primary carriers 10 which are attached to the surface area 24a of the sensor electrode device 20 serving as secondary carrier.

In the practical example shown in FIG. 4 in diagrammatic form not true to scale, only a single primary carrier 10 is shown. This consists of a lipid vesicle or liposome in the form of a lipid double layer or lipid membrane 11 formed as an essentially hollow closed sphere. In this lipid double layer 11 of the vesicle serving as primary carrier 10, a membrane protein is embedded in a manner penetrating through the membrane as essentially biological unit 12.

By converting a substrate S present in the measuring medium 30 into a converted substrate S′, certain processes are initiated in the membrane protein 12 which, in the case shown in FIG. 1, leads to a substance transport of a species Q from the extra-vesicular side or outside 10a of the vesicle 10 to the intravesicular side or inside 10b of the vesicle 10. If the species Q has an electric charge, the transportation of the species Q from side 10a to side 10b leads to a net charge transportation which corresponds to an electric current from the outside 10a of the vesicle 10 to the inside 10b of the vesicle 10.

Into each vesicle 10, a multiplicity of essentially identical membrane protein molecules 12 are incorporated in essentially the same orientation into membrane 11 of the vesicle 10 as a rule and on the one hand. If these are essentially simultaneously activated—e.g. by a concentration jump, initiated by mixing, in the concentration of the substrate S of a non-activating measuring medium N, 30 without substrate S to an activating measuring medium A, 30 with substrate S—this leads to a measurable electric current.

This charge carrier transportation is measurable because a multiplicity of primary carriers 10 or vesicles are attached to the surface 24a of the sensor electrode device 20 such that, on activation of a multiplicity of protein molecules 12 in a multiplicity of vesicles in front of the surface 24a of the sensor electrode device 20, a spatial charge of a certain polarity is formed. This spatial charge then acts onto the electrode 26 which, in the case shown in FIG. 1, is vapour deposited onto a carrier 22 of glass in the form of a gold layer and covered by a double layer, serving as insulation area 24, of a bottom layer 24b and a top layer 24a serving as surface and electrically insulated vis-à-vis the measuring medium 30.

The surface or upper layer 24a of the insulation area 24 is a lipid monolayer, for example, which is compatible with the lipid double layer 11 of the vesicle 10 which monolayer is formed by means of a self-assembly process on an alkane thiol monolayer forming the bottom layer 24b in such a way that the sequence of the layers 24b and 24a, namely the sequence of an alkane thiol monolayer and a lipid monolayer, forms a membrane structure SSM as electrode 26 on a gold substrate formed in the manner of a solid body, which membrane structure is also referred to as solid supported membrane (SSM).

The sensor arrangement 1 and, in particular, the sensor electrode device 20 is connected to a data acquisition/control device 40 via a connecting line 48i. This device is equipped with a measuring device 44 in which an electric current I(t) or an electric voltage U(t) can be measured as a function of time. Moreover, an amplifier device 42 is anticipated in which the measuring signals are filtered and/or amplified. Via a control line 48s, the active principle testing is controlled by controlling the exchange/measuring device 60. Via a further line 48o, the electric circuit is closed by a counter-electrode 46, e.g. in the form of a Pt/Pt electrode or by an Ag/AgCl electrode. Insulations 28, 27 and 47 prevent short circuits of the SSM and/or the counter-electrode 46 vis-à-vis the measuring medium 30.

FIG. 5 shows a diagrammatic and partly sectional side view of an embodiment of the sensor arrangement 1 according to the invention in the case of which a membrane fragment 10 is provided as primary carrier 10 instead of a vesicle or liposome, into which fragment a membrane protein is embedded as biological unit 12 in an oriented manner. With respect to the embodiment of FIG. 5, it should be noted that the representation is not true to scale and on the other hand, a large plurality of membrane fragments are, as a rule, attached or adsorbed simultaneously to the SSM or the surface 24a of the sensor electrode device 20 serving as secondary carrier.

Here, too, it is shown that, by converting the substrate S provided in the measuring medium 30 into a converted substrate S′, a substance transport of the species Q from one side 10a of the membrane fragment 10 to the opposite side 10b takes place which can be detected via the corresponding net charge transport and the displacement current connected therewith as a function of the time.

The invention has been described with particular reference to the preferred embodiments thereof, but it should be understood that variations and modifications within the spirit and scope of the invention may occur to those skilled in the art to which the invention pertains.

1. A biocompatible sensor electrode arrangement comprising:
at least one carrier substrate area having a top side with a surface area;
at least one intermediate substrate area formed on the surface area of the carrier substrate area or a part thereof in a structured manner and formed with a top side facing away from the carrier substrate area with a surface area; and
a biomaterial area formed on the surface area of the intermediate substrate area or a part thereof in a structured manner, with at least one biologically compatible material component,
the carrier substrate with the intermediate substrate area thereon or the intermediate substrate area as such or a part thereof in each case being formed in the form or the manner of a wafer element or a printed circuit;
the intermediate substrate area being provided as at least one electrically conductive electrode of the electrode area;
the biomaterial area being provided as an electrically insulating insulation area; and
in operation, the electrode concerned being electrically insulated by the biomaterial area from a measuring medium, from the primary carriers and from the biological units;
the biomaterial area being formed as layers;
the biomaterial area being formed at least partly of a sequence of monolayers, comprising a sub-layer and a top layer;
the monolayers being formed as spontaneously self-organising layers;
as said sub-layer of the biomaterial area, a layer of a long-chain alkane thiol being provided as bottom most area facing towards the electrode of the biomaterial area; and
as said top layer of the biomaterial area, a layer of a lipid being provided as an uppermost area facing away from the electrode or surface area of the biomaterial area.

2. The sensor electrode arrangement according to claim 1, wherein the carrier substrate area exhibits a chemically inert, biologically inert and electrically insulating material or is formed of such a material.

3. The sensor electrode arrangement according claim 1, wherein the carrier substrate area comprises a mechanically flexible material or is formed as such.

4. The sensor electrode arrangement according to claim 1, wherein a metallic layer structure is formed on the surface of the carrier substrate area for the intermediate substrate area.

5. The sensor electrode arrangement according to claim 4, wherein the layer structure for the intermediate substrate area comprises at least one or of at least one primary metal area arranged bottom most, a subsequent auxiliary layer and an actual electrode layer arranged top most.

6. The sensor electrode arrangement according to claim 5, wherein the primary metal area is formed with or of copper.

7. The sensor electrode arrangement according to claim 5, wherein the primary metal area is formed by a process selected from the group consisting of photolithographically, bonded on, laminated on and printed on.

8. The sensor electrode arrangement according to claim 5, wherein the auxiliary layer is formed with or of nickel.

9. The sensor electrode arrangement according to claim 5, wherein the actual electrode layer arranged uppermost is formed with or of a noble metal.

10. The sensor electrode arrangement according to claim 5, wherein at least one of the auxiliary layer and the actual electrode layer arranged uppermost are formed by electrodeposition.

11. The sensor electrode arrangement according to claim 1, wherein the carrier substrate area is formed entirely or partly of a chemically inert, biologically inert material and a material at most slightly absorptive vis-à-vis proteins, biologically and chemically active principles.

12. The sensor electrode arrangement according to claim 1, wherein the carrier substrate area is formed with or of a material selected from the group consisting of PMMA, PTFE, POM, FR4, polyimide, PI, Kapton, PEN, PET and materials transparent in the UV range.

13. The sensor electrode arrangement according to claim 1, wherein a plurality of identical intermediate substrate areas and biomaterial areas is formed electrically insulated from each other and are laterally arranged side by side on the carrier substrate area.

14. The sensor electrode arrangement according to claim 13, wherein the plurality of intermediate substrate areas and biomaterial areas are arranged in sequence or in matrix form.

15. The sensor electrode arrangement according to claim 1, wherein said sensor electrode arrangement is formed as a sensor electrode arrangement for at least one of amperometric, potentiometric, pharmacological active site and active principle testing.

16. The sensor electrode arrangement according to claim 1, wherein the intermediate substrate area and the biomaterial area are each provided in a form selected from the group consisting of a membrane biosensor electrode area and a secondary carrier of the sensor electrode arrangement.

17. The sensor electrode arrangement according to claim 1, wherein the intermediate substrate area and the biomaterial area are each provided in a form selected from the group consisting of a membrane biosensor electrode area and a secondary carrier with an electrically conductive and solid body-type electrode area.

18. The sensor electrode arrangement according to claim 17, wherein a plurality of primary carriers is provided in immediate spatial vicinity of the secondary carrier, the primary carriers being activable to electronic action and biological action.

19. The sensor electrode arrangement according to claim 18, wherein
as primary carrier, a primary carrier from the group is provided comprising a eukaryotic cell, a prokaryotic cell, a bacterium, a virus, components, membrane fragments, or associations thereof in the native form or in a modified form or
as primary carrier, a primary carrier of the group is provided comprising a vesicle, a liposome or a micellar structure.

20. The sensor electrode arrangement according to claim 15, wherein the area insulating and covering the electrode, of the biomaterial area or the insulation area comprises a membrane structure with a surface of approximately A≈0.1-50 mm² and with a specific electric conductivity of approximately Gₘ≈1-100 nS/cm² and/or with a specific capacitance of approximately Cₘ≈10-1000 nF/cm².

21. The sensor electrode arrangement according to claim 15, further comprising a biological unit activable to electrogenic charge carrier movement or to electrogenic charge carrier transportation.

22. The sensor electrode arrangement according to claim 15, further comprising a biological unit selected from the group consisting of a membrane protein, an ion pump, an ion channel, a transporter, a receptor, a component and an association thereof.

23. The sensor electrode arrangement according to claim 22, wherein the biological unit is provided in the native form or in a form selected from the group consisting of a modified form, a purified form, a form modified microbiologically and a form modified by molecular biology.

24. The sensor electrode arrangement according to claim 15, wherein,
the surface of the primary carriers and the surface of the secondary carrier comprise an opposite polarity or charge or a connection of the type of a chemical bond being formed via a His-Tag coupling or a streptavidin biotin coupling,
between the surface of the primary carriers and the surface of the secondary carrier.

25. A process for manfacturing a biocompatible sensor electrode arrangement comprising the steps of:
forming at least one carrier substrate area with a top side having a surface area;
forming at least one intermediate substrate area on the surface area of the carrier substrate area or a part thereof in a structured manner and with a top side facing away from the carrier substrate area with a surface area; and
forming a biomaterial area on the surface area of the intermediate substrate area or a part thereof in a structured manner with at least one biologically compatible material component;
the carrier substrate area with the intermediate substrate area thereon or the intermediate substrate area as such or a part thereof being formed in the form or in the manner of selected from the group consisting of a wafer element and a printed circuit;
the intermediate substrate area being provided as at least one electrically conductive electrode of the electrode area;
the biomaterial area being provided as an electrically insulating insulation area; and
in operation the biomaterial area electrically insulating the electrode from a measuring medium, from the primary carriers and from the biological units;
the biomaterial area being formed as layers;
the biomaterial area being formed at least partly of a sequence of monolayers, comprising a sub-layer and a top layer;
the monolayers being formed as spontaneously self-organising layers;
as said sub-layer of the biomaterial area, providing a layer of a long-chain alkane thiol as a bottom most area facing towards the electrode of the biomaterial area; and
as said top layer of the biomaterial area, providing a layer of a lipid as an uppermost area facing away from the electrode or surface area of the biomaterial area.

26. The process according to claim 25, comprising the steps of forming
the carrier substrate with the intermediate substrate area thereon or the intermediate substrate area as such or a part thereof:
as a or with a photolithographically processed structure or as a or with a photographically processed element;
as a or with a structure being bonded on or laminated on or as an or with an element processed by being bonded on or laminated on;
as a or with a structure processed by at least one of micromechanically and laser ablation or as an or with an element processed by at least one of micromechanically and laser ablation; and/or
as a or with a structure processed by printing or as an or with an element processed by printing on the carrier substrate.

27. The process according to claim 25, wherein the carrier substrate area is formed with or of a material selected from the group consisting of a chemically inert material, a biologically inert material and an electrically insulating material.

28. The process according to claim 25, wherein the carrier substrate area is formed with a mechanically flexible material or of such a material.

29. The process according to claim 25, further comprising the step of forming a metallic layer structure on the top side surface of the carrier substrate area for the intermediate substrate area.

30. The process according to claim 29, wherein the layer structure for the intermediate substrate area or for the connecting substrate layer is formed with at least one or of at least one primary metal area arranged bottom most, a subsequent auxiliary layer as an alloy and/or diffusion barrier and an actual electrode layer arranged top most.

31. The process according to claim 30, wherein the primary metal area comprises copper.

32. The process according to claim 31, comprising the step of forming primary metal area by a process selected from the group consisting of photolithographically, bonded on, laminated on and printed on.

33. The process according to claim 32, wherein the auxiliary layer comprises nickel.

34. The process according to claim 30, wherein the actual electrode layer arranged top most comprises a noble metal.

35. The process according to claim 30, wherein at least one of the auxiliary layer and the actual electrode layer arranged top most are formed by electrodeposition.

36. The process according to claim 25, wherein to the carrier substrate area is formed entirely or partly of at least one material selected from the group consisting of a chemically inert material, a biologically inert material and a material at most slightly absorptive vis-à-vis proteins, biologically and/or chemically active principles.

37. The process according to claim 25, wherein the carrier substrate area is formed entirely or partially of a material selected from the group consisting of PMMA, PTFE, POM, FR4, polyimide, PEN, PET and a material which is transparent in the UV range.

38. The process according to claim 25, comprising the step of forming a plurality of intermediate substrate areas or connecting substrate areas and/or biomaterial areas in a connected or in a separated form.

39. The process according to claim 38, wherein the plurality of the intermediate substrate areas and/or biomaterial areas are arranged in sequence or in matrix form.

40. The process according to claim 25, comprising the step of forming the sensor electrode arrangement as a sensor electrode arrangement for amperometric and/or potentiometric, pharmacological active site and/or active principle testing.

41. The process according to claim 40, further comprising the step of providing at least one of the intermediate substrate area and the biomaterial area in a form selected from the group consisting of a membrane biosensor electrode area and a secondary carrier of the sensor electrode arrangement.

42. The process according to claim 41, further comprising the step of forming the intermediate substrate area and the biomaterial area in a form selected from the group consisting of a membrane biosensor electrode area and a secondary carrier with an electrically conductive and solid body-type electrode area.

43. The process according to claim 42, further comprising the step of providing a plurality of primary carriers in immediate spatial vicinity of the secondary carrier, the primary carriers containing units which are activable to electronic action and biological action.

44. The process according to claim 43, further comprising the step of providing said
primary carriers as a primary carrier comprising a eukaryotic cell, a prokaryotic cell, a bacterium, a virus or components, membrane fragments, or associations thereof in the native form or in a modified, purified form or a form modified microbiologically or by molecular biology or
in which, as primary carrier, a primary carrier comprising a vesicle, a liposome or a micellar structure.

45. The process according to claim 39, comprising the step of forming the area, insulating and covering the electrode, of the biomaterial area or the insulation area with a membrane structure (SSM) with a surface of approximately A≈0.1-50 mm² and with a specific electric conductivity of approximately Gₘ≈1-100 nS/cm² and/or with a specific capacitance of approximately Cₘ≈10-1000 nF/cm².

46. The process according to claim 39, comprising the step of providing biological unit which is activable to electrogenic charge carrier movement.

47. The process according to claim 39, comprising the step of providing a membrane protein as a biological unit.

48. The process according to claim 47, comprising the step of providing the biological unit in a form selected from the group consisting of the native form and a modified form.

49. The process according to claim 39, comprising at least one of the following steps of forming
the surface of the primary carriers and the surface of the secondary carrier formed with an opposite polarity or charge and
forming a chemical bond connection between the surface of the primary carriers and the surface of the secondary carrier.

50. The sensor electrode arrangement according to claim 3, wherein the carrier substrate area is in the form or the manner of a film.

51. The sensor electrode arrangement according to claim 5, wherein said subsequent auxiliary layer is at least one of an alloy and a diffusion barrier.

52. The sensor electrode arrangement according to claim 9, wherein said noble metal is gold.

53. The sensor electrode arrangement according to claim 18, wherein said primary carriers are activatable to membrane proteins.

54. The sensor electrode arrangement according to claim 19, wherein as primary carrier, said primary carrier from the group is provided in a form selected from the group consisting of the purified form and a form modified microbiologically and by molecular biology

55. The process according to claim 28, wherein the carrier substrate area is in the form or manner of a film.

56. The process according to claim 34, wherein said noble metal is gold.

57. The process according to claim 37, wherein said polyimide is selected from the group consisting of PI and Kapton.

58. The process according to claim 38, comprising the step of forming said plurality of intermediate substrate areas or connecting substrate areas and/or biomaterial areas in a form electrically insulated from each other, wherein said intermediate substrate areas are identical.

59. The process according to claim 43, wherein said primary carriers containing units are activable to biological membrane proteins.

60. The process according to claim 46, comprising the step of providing said biological unit which is activable to electrogenic charge carrier transportation.

61. The process according to claim 47, wherein said membrane protein is selected from the group consisting of an ion pump, an ion channel, a transporter, a receptor and a component or an association thereof.

62. The process according to claim 48, comprising the step of providing the biological unit in a form selected from the group consisting of a purified form, a form modified microbiologically and a form modified by molecular biology.

63. The process according to claim 49, wherein said chemical bond is selected from the group consisting of a His-Tag coupling and a streptavidin biotin coupling.
